(19) United States
(12) Patent Application Publication
(10) Pub. No.: US 2010/0145429 A1
(43) Pub. Date: Jun. 10, 2010

(54) Introducer sheath and method of manufacture

(75) Inventors: Manjiri A. Dhoke, Bloomington, IL (US); William L. Howat, Laconia, NH (US); Cleve Koehler, Elletsville, IN (US); Fred T. Parker, Unionville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/316,114

(22) Filed: Dec. 9, 2008

(51) Int. Cl. A61F 2/84; B29C 59/00; B32B 37/00

(52) U.S. Cl. 623/1.11; 264/320; 156/173

(57) ABSTRACT

The present invention relates to a medical device delivery system. The delivery system includes an introducer sheath having an elongated tubular member having a passageway extending generally longitudinally therethrough. The passageway is defined by an inner surface of the tubular member and is configured to receive a medical device moveably disposed therein, where at least a portion of the inner surface of the inner tube comprises a non-uniform surface configured to reduce surface contact between the inner surface and the medical device. The delivery system includes an inner catheter configured to slidably extend throughout the passageway of the introducer sheath. A prosthesis is disposed within a distal portion of the introducer sheath, where the prosthesis is supported within the introducer sheath by the inner catheter.

### BACKGROUND

1. Technical Field

This invention relates generally to medical devices and, in particular, to a delivery catheter or sheath and, more particularly, to an introducer sheath having a non-uniform surface within the inner surface of the distal end of the introducer sheath.

2. Background Information

Introducer catheters or sheaths are widely used to provide a conduit for percutaneous access to the vascular system. Such sheaths are generally of thin-wall construction, and thus, have a tendency to kink when traversing within the narrow confines of the vascular system. Increasing the thickness of the sheath wall minimally improves the level of kink resistance, however this level is still often considered unacceptable. In addition, increasing the thickness of the sheath wall is generally considered undesirable, because it necessitates the use of a larger entry hole than would otherwise be required.

Introducer sheaths are widely used for delivering an implantable medical device, such as a stent or a stent graft, to a deployment site well within the vasculature of the patient. For example, an introducer sheath may be used to maintain a self-expanding prosthesis in a compressed configuration for delivery to the deployment site. The prosthesis is disposed within a distal portion of the introducer sheath, and is supported by an inner catheter, or pusher, that slidably extends through the interior lumen of the introducer sheath. During deployment, the pusher maintains the longitudinal position of the prosthesis as the introducer sheath is withdrawn to expose the prosthesis from the distal end of the introducer sheath. Catheters or sheaths used to deliver such devices are likewise susceptible to kinking, particularly when the implantable medical device or pusher does not have a uniform diameter to support the delivery catheter or sheath along its entire length. Furthermore, deployment of a medical device, e.g. prosthesis, using conventional introducer sheaths provides a problem due to extremely high levels of deployment forces during delivery of the stent. This high amount of force is caused by the amount of friction present between the interior surface of the introducer sheath and the medical device.

In an effort to reduce kinking, introducer catheters and sheaths have been developed that include a metal reinforcing coil or braid disposed within the wall, such as disclosed in U.S. Pat. Nos. 5,380,304 and 5,700,253, the entire contents of which are hereby incorporated by reference. Although these types of sheaths are less prone to kinking, the rigidity tends to increase the frictional deployment forces in prosthesis delivery systems. Therefore, it is desired to provide an introducer sheath that provides sufficient resistance to kinking while reducing the amount of contact points between a medical device and an introducer sheath in order to reduce the effects of friction during deployment of the device.

### BRIEF SUMMARY

In one aspect of the present invention, a medical device delivery system for a prosthesis including an introducer sheath having an elongated tubular member having a passageway extending generally longitudinally therethrough. The passageway is being defined by an inner surface of the tubular member and is configured to receive a medical device moveably disposed therein, where at least a portion of the inner surface comprises a non-uniform surface configured to reduce surface contact between the inner surface and the medical device. The delivery system also includes an inner catheter configured to slidably extend throughout the passageway of the introducer sheath, and a prosthesis disposed within a distal portion of the introducer sheath, wherein the prosthesis is supported within the introducer sheath by the inner catheter.

In another aspect of the present invention, an introducer sheath includes an elongated tubular member having a passageway extending generally longitudinally therethrough. The passageway is defined by an inner surface of the tubular member and is configured to receive a medical device moveably disposed therein, where at least a portion of the inner surface of the inner tube comprises a non-uniform surface configured to reduce surface contact between the inner surface and the medical device.

In still another aspect of the present invention, a method of providing non-uniform surface to an introducer sheath includes providing an elongated tubular member having a passageway extending generally longitudinally therethrough. A non-uniformity is formed on a surface on a distal portion of an inner surface of the elongated tubular member configured to reduce surface contact between a medical device and the inner surface.

Other systems, methods, features, and advantages will be, or will become, apparent to one with skill in the art upon examination of the following figures and detailed description. It is intended that all such additional systems, methods, features, and advantages be included within this description, be within the scope of the disclosure, and be protected by the following claims.

### BRIEF DESCRIPTION OF THE DRAWINGS

The introducer sheath and method of manufacture of the present invention may be understood with reference to the following drawings and description. The components in the figures are not necessarily to scale, emphasis instead being placed upon illustrating the principles of the disclosure. Moreover, in the figures, like referenced numerals designate corresponding parts throughout the different views.

FIG. 1 depicts a cross-sectional side view of an embodiment of a delivery system of the present invention;

FIG. 2 depicts a cross-sectional end view of the delivery system shown in FIG. 1 on line 2-2;

FIG. 3 depicts a cross-sectional side view of the manufacture of an embodiment of the introducer sheath of the present invention;

FIG. 4 depicts a cross-sectional perspective view of an embodiment of the introducer sheath of the present invention;

FIG. 5 depicts a cross-sectional perspective view of an alternative embodiment of the introducer sheath of the present invention;

FIG. 6 depicts a cross-sectional perspective view of the introducer sheath of FIG. 3, where a stent is disposed within the passageway of the inner tube;

FIG. 7 depicts a cross-sectional perspective view of the introducer sheath of FIG. 4, where a stent is disposed within the passageway of the inner tube;

FIGS. 8A-8C depict perspective views of profiled mandrels used to form alternate embodiments of the introducer sheath of the present invention; and

FIG. 9 depicts a cross-sectional perspective view of the distal end of an alternative embodiment of the introducer sheath of the present invention, where a stent is disposed within the passageway of the inner tube;

Further advantages, as well as details of the present invention ensue from the following description of the attached drawings.

### DETAILED DESCRIPTION OF THE DRAWINGS AND THE PRESENTLY PREFERRED EMBODIMENTS

The present invention relates to a medical device delivery system. The delivery system includes an introducer sheath having an elongated tubular member having a passageway extending generally longitudinally therethrough. The passageway is defined by an inner surface of the tubular member and is configured to receive a medical device moveably disposed therein, where at least a portion of the inner surface of the inner tube comprises a non-uniform surface configured to reduce surface contact between the inner surface and the medical device. The delivery system includes an inner catheter configured to slidably extend throughout the passageway of the introducer sheath. A prosthesis is disposed within a distal portion of the introducer sheath, where the prosthesis is supported within the introducer sheath by the inner catheter.

Unless defined otherwise, all technical and scientific terms used herein have the same meaning as commonly understood to one of ordinary skill in the art to which this invention belongs. In case of conflict, the present document, including definitions, will control. Preferred methods and materials are described below, although methods and materials similar or equivalent to those described herein can be used in the practice or testing of the present disclosure. All publications, patent applications, patents, and other references mentioned herein are incorporated by reference. The materials methods, and examples disclosed herein are illustrative only and not intended to be limiting.

The term “medical device” means any object that is itself or that includes a component that is intentionally inserted into the body of a patient as part of a medical treatment, and that comprises a structure adapted for introduction into a patient. The medical device can be a tool, such as, without limitation, a catheter, a wire guide, a foreceps, or a scissors used to affect a surgical procedure at and/or deliver a second medical device to a treatment site in a patient. An alternative medical device of the present invention is one that is commonly intended to be a permanent implant, such as, for example, a graft or a stent.

The term “prosthesis” means any replacement for a body part or function of that body part. It can also mean a device that enhances or adds functionality to a physiological system. Such prostheses may include, but are not limited to, self-expanding devices, balloon-expandable devices, non-expandable devices, heart valve prostheses, and venous valve prostheses.

The term “stent” means any device or structure that adds rigidity, expansion force, or support to a prosthesis. A stent is used to obtain and maintain the patency of the body passageway while maintaining the integrity of the passageway. Also, the stent may be used to form a seal. The stent may be coated with a polymeric material or a drug, for example, by immersion in molten polymer or any other method known to one of skill in the art. The stent may be located on the exterior of the device, the interior of the device, or both. A stent may be self-expanding, balloon-expandable or may have characteristics of both. A variety of other stent configurations are also contemplated by the use of the term “stent.”

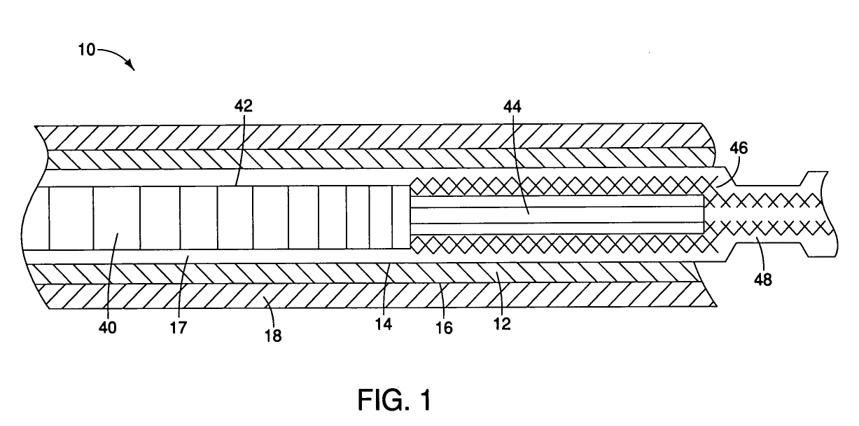

FIG. 1 depicts an exemplary delivery system according to the present invention. The delivery system 10 includes an introducer sheath 12 configured to maintain a medical device 30, such as a prosthesis, in a compressed configuration for delivery to a deployment site within the body of the patient. A prosthesis, such as a stent, is used to obtain and maintain the patency of the body passageway while maintaining the integrity of the passageway. The stent may be self-expanding or balloon-expandable, and may be deployed according to conventional methodology, such by an inflatable balloon catheter, by a self-deployment mechanism (after release from a catheter), or by other appropriate means. Preferred materials include those materials that can provide the desired functional characteristics with respect to mechanical load bearing, biological compatibility, modulus of elasticity, or other desired properties. In various embodiments, the stent includes a metallic material selected from stainless steel, nickel, silver, platinum, palladium, gold, titanium, tantalum, iridium, tungsten, cobalt, chromium, a molybdenum alloy, a molybdenum alloy including about 0.4% to about 0.8% of lanthanum oxide (Li₂O₃), a nickel-titanium alloy, or a superelastic nickel-titanium (NiTi) alloy. The stent may be any suitable vascular stent such as the commercially available Gianturco-Roubin FLEX-STENT®, GRII TM, SUPRA-G, ZILVER, OR V FLEX coronary stents from Cook Incorporated (Bloomington, Ind.).

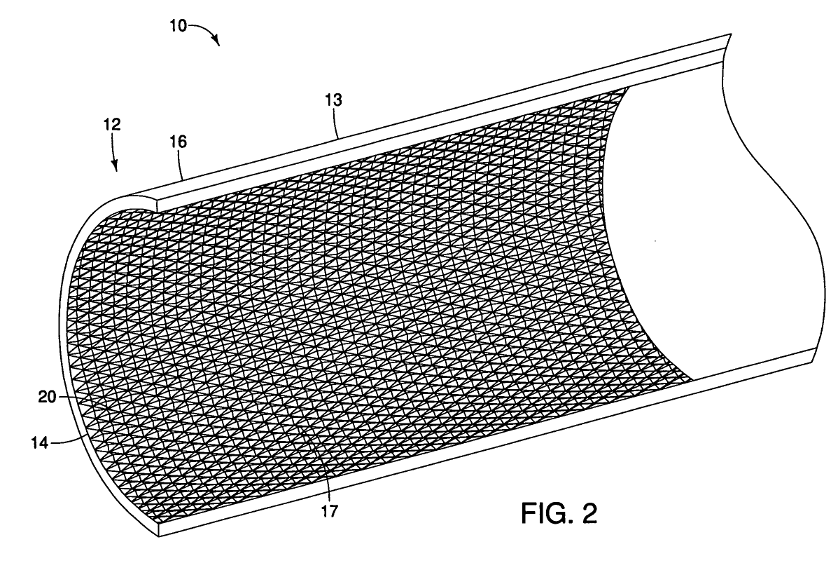

The introducer sheath 12 includes a tube 14 having an inner surface 16 and an outer surface 18. The stent 30 is disposed within the distal portion of the introducer sheath 12 and is supported by an inner catheter 24, or a pusher member. The inner catheter 24 is slideably positioned within the interior passageway 17 of the introducer sheath 12. During deployment, the inner catheter 22 maintains the longitudinal position of the stent 30 as the introducer sheath 12 is withdrawn to expose the prosthesis from the distal end of the introducer sheath. The introducer sheath 12 may be introduced over a wire guide 26. The wire guide 26 is inserted in the vessel with an introducer needle using, for example, the well-known percutaneous vascular access Seldinger technique. As shown by FIGS. 1 and 2, the distal portion of the introducer sheath 12 includes a non-uniform surface 20 upon the inner surface 16 of the tube 14. As will be discussed in further detail below, the non-uniform surface 20 is configured to reduce contact between the outer surface of the stent 30 and the inner surface 14 of the introducer sheath 12.

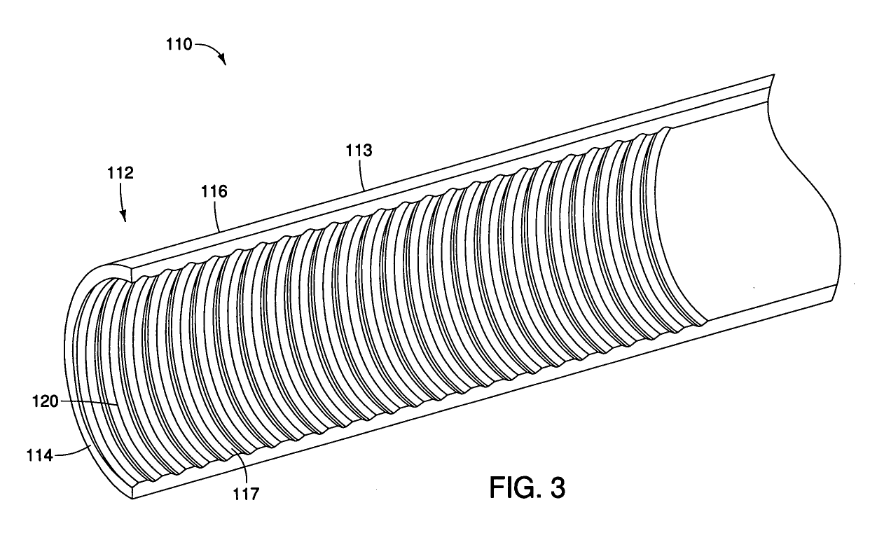

FIG. 3 depicts an exemplary method of manufacture of an embodiment of an introducer sheath 12 of the present invention. The present invention may be used with different types of introducer sheaths. One such acceptable introducer sheath that could be used with this invention is the Flexor® sheath manufactured by Cook Incorporated, Bloomington, Ind. The introducer sheath 12 includes an inner tube 14 and an outer tube 28 mechanically connected to an outer surface 18 of the inner tube 14. The distal end of the introducer sheath 12 is configured to receive a prosthesis, such as a stent or a stent-graft. In one embodiment, the inner tube 14 is about 2.91″ in length of a lubricous material tube such as polytetrafluoroethylene having a uniform inside diameter in the range of about 0.0825″ to about 0.0840″ with a wall thickness of 0.0015″ plus or minus 0.0005″ before heating. The inner tube 14 may have a minimum inside dimension of 0.081″ after heating. Generally, the lubricous polytetrafluoroethylene material presents a slippery inner surface 16 for the easy insertion and withdrawal of the dilator as well as other catheters and medical apparatus. The outer surface 18 of the inner tube 14 may be chemically etched in a well-known manner for forming a rough outer surface 18 to which the outer tube 28 is mechanically connected using a well-known heat shrinking and formation process. A reinforcement material, such as a coil, may be compression fitted upon the outer surface 18 of the inner tube 14 to help prevent any kinking of the introducer sheath during deployment of the stent.

The outer tube 28 may be about 2.91″ in length with an inside diameter of 0.103″ plus or minus 0.02″ of a heat formable polyamide material such as nylon that is heat shrunk through the turn spacings of the coil. The wall thickness of the nylon tube is approximately 0.065″ plus or minus 0.01″. After the outer tube 28 is heat shrunk onto the rough outer surface 18 of the inner tube 14, the shrink tube is removed therefrom, and a taper may be formed at the distal end of the sheath 12. The tapered distal end of the sheath 12 is formed by cutting and slitting a about a 0.118″ length of nylon tubing having about a 0.100″ inside diameter and inserting it into a well-known taper mold. The distal end of inner tube 14 may vary along the length of the tapered distal end of the outer tube 28, but should not extend all the way to the distal end of the outer tube so as not to break the tapered surface of the outer tube 28. The distal end of the sheath 12 may be formed into a flared configuration in a well-known manner such as inserting over a heated, tapered tip end and then cooled.

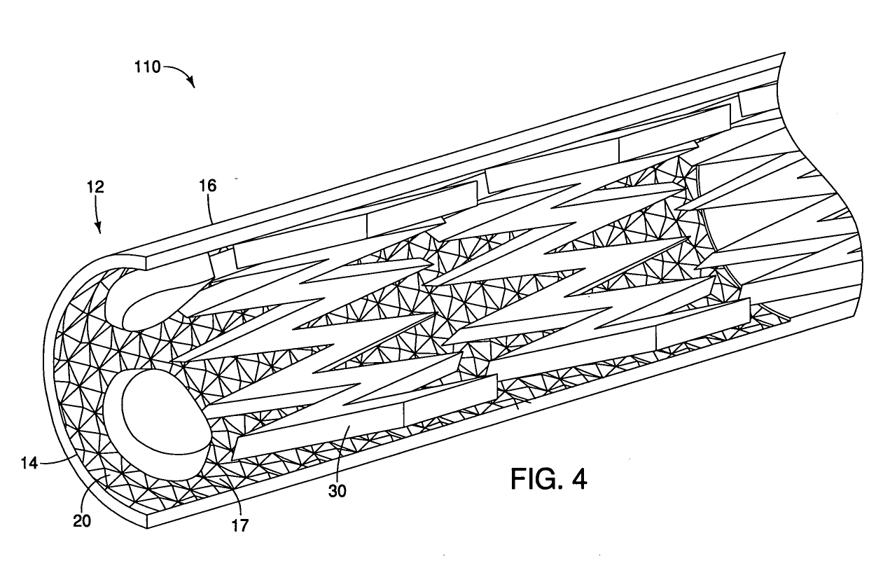

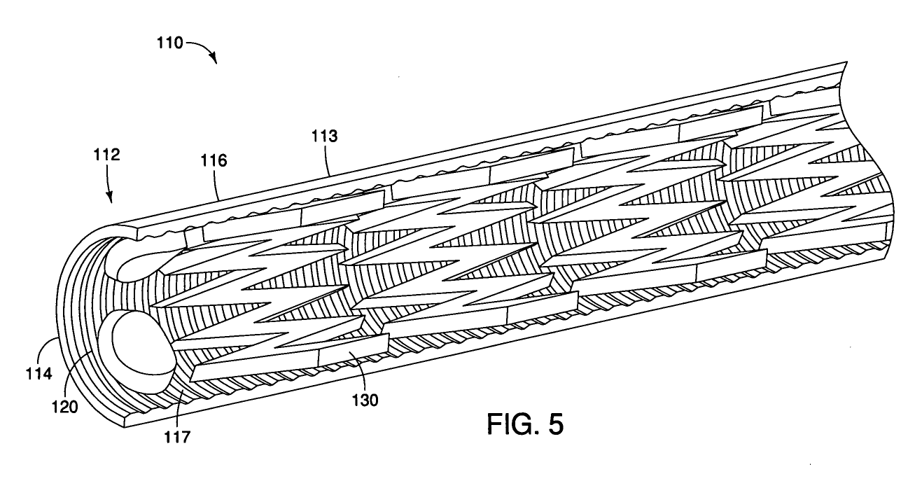

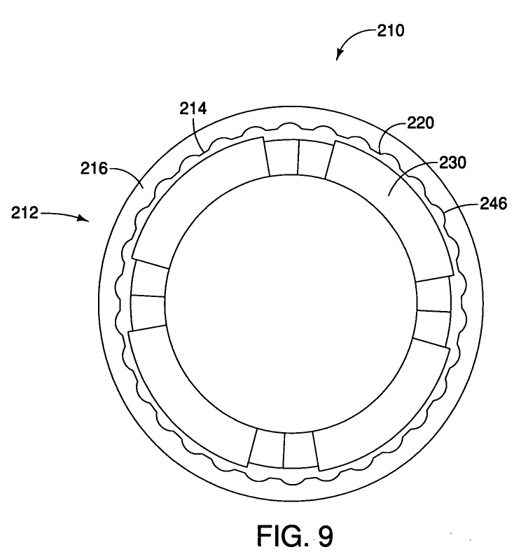

FIG. 4 shows the distal end of the inner tube 14 of this embodiment of the introducer sheath 12 in further detail. The distal end includes a non-uniform surface 20 upon the inner surface 16 of the inner tube 14. The non-uniform surface 20 provides advantages during the actual deployment a stent from the introducer sheath 12 by promoting less surface contact between the inner surface 16 of the inner tube 14 and the stent. The size of non-uniform surface 20 is based on the size of the stent which will be deployed within the body of the patient. The non-uniform surface 20 may encompass multiple configurations. In this embodiment, the non-uniform surface 20 has a braided configuration. FIG. 5 shows another embodiment of the introducer sheath 112 where the non-uniform surface 120 of the inner surface 116 of the inner tube 114 has a spiral configuration. In other embodiments, the non-uniform surface 220 of the inner surface 216 of the inner tube 214 has a plurality of splines, as shown by FIG. 9. As will be discussed in further detail below, the different configurations may be formed by introducing a wire having a braided, spiral, or spline configuration during the manufacturing process for the introducer sheath 12.

Referring back to FIG. 3, a mandrel 40 having a proximal end 42 and a distal end 44 is introduced during the manufacture of the introducer sheath 10. The mandrel 40 is sized to fit within a passageway 17 formed by the inner tube 14. The distal end 44 of the mandrel 40 is configured to have a reduced diameter from the proximal end 42 in order to provide space for the introduction of the wire material to help form the non-uniform surface 20. The length of reduced diameter of the distal end 44 of the mandrel 40 is for at least about 5.1″ of the mandrel 40. In this particular embodiment, the braided configuration of the non-uniform surface 20 is achieved by introducing a wire braid 46 around the distal end 44 of the mandrel 40. The ends of the wire braid 46 are configured around the distal end 44 of the mandrel 40 such that they hang loosely over the end of the mandrel 40. In some embodiments, a spiral wire may be placed about the distal end of mandrel 40 instead of the wire braid 46 in order to create the spiral configuration shown in FIG. 5. Wires having other configurations may also be introduced during the manufacturing process in order to create the non-uniform surface.

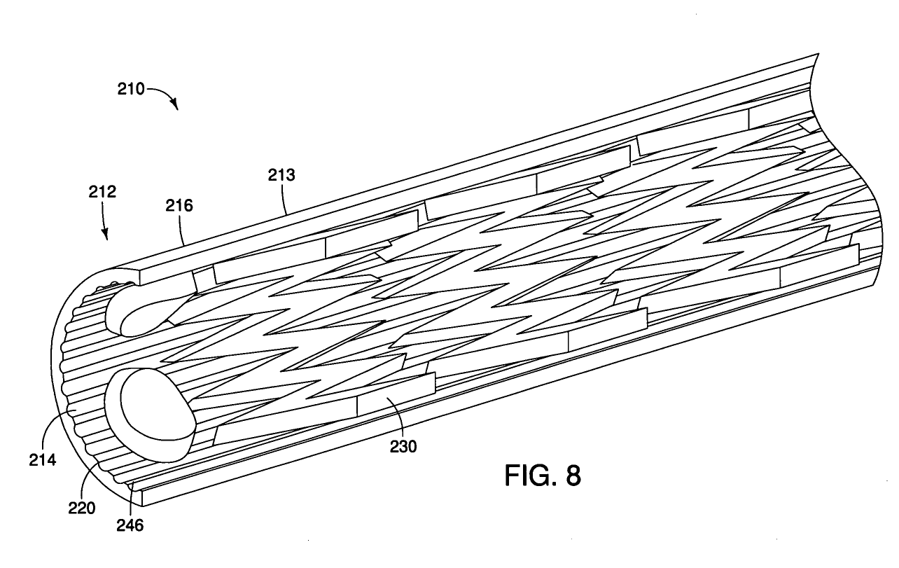

In other embodiments, a pattern having the desired configuration may be formed on the surface of the distal end 44 of the mandrel 40. The pattern disposed on the distal end 44 of the mandrel 40 extends away from the mandrel 40 in the radial direction, and its depth would be limited by the compression diameter of the prosthesis. As shown by FIGS. 8A-8C, numerous different patterns may be cut into the distal portion of the mandrel to form the non-uniform surface of the introducer sheath. FIG. 8A shows an embodiment of a mandrel 340 where a knurl pattern 346 has been cut into the distal end 44. FIG. 8B shows an embodiment of a mandrel 240 where splines 246 have been cut into the distal end 244. Referring now to FIG. 9, an embodiment of the inner tube 212 is shown where the shape of the splines 246 from the mandrel is transferred onto the inner surface 216 of the inner tube 214. FIGS. 8C shows an embodiment of a mandrel 140 where a spiral pattern 146 has been cut into the distal end 144. Other patterns may be suitable for use with the present invention.

FIG. 3 shows a heat shrink tube 48 is positioned about the entire surface of the mandrel 40. As with the wire braid 46, the heat shrink tube 48 is also positioned such that the ends of the heat shrink tube 48 hang loosely over the end of the mandrel 40. As stated above, an inner tube 14, a coil, and an outer tube 28 are positioned about the remaining portions of the introducer sheath 10 are formed about the mandrel 40. The coil is compression fitted by collapsing inner tube 14 and inserting the wire coil there over. Inner tube 14 is then compressed-air expanded to engage and compression fit the inner surface of the flat wire coil. The outer tube 28 is heated and compressed through the spaces between the coil turns with a heat shrink tube for mechanically connecting to outer surface 18 of the inner tube 14. As a result, the outside diameter of the outer tube 28 is approximately 0.22″ greater than that of the inner tube 14.

As the heat shrink tube 48 surrounding the distal end 44 of the mandrel 40 shrinks, the heated inner tube 14 is compressed about the wire braid 46 to mechanically create the non-uniform, braided surface 20 on the inner surface 16 of the inner tube 14. This braided surface impression is present on both the heat shrink tube 48 and the inner surface 16 of the inner tube 14. The wire braid 46 is removed from the passageway 17 of the inner tube 14 after heating the introducer sheath 10. Thus, only the non-uniform, braided surface 20 remains within the inner surface 16 of the inner tube 14. As stated above, the non-uniform surface 20 decreases the amount of contact points between the outer surface of the stent and the inner surface of the inner tube during pre-deployment.

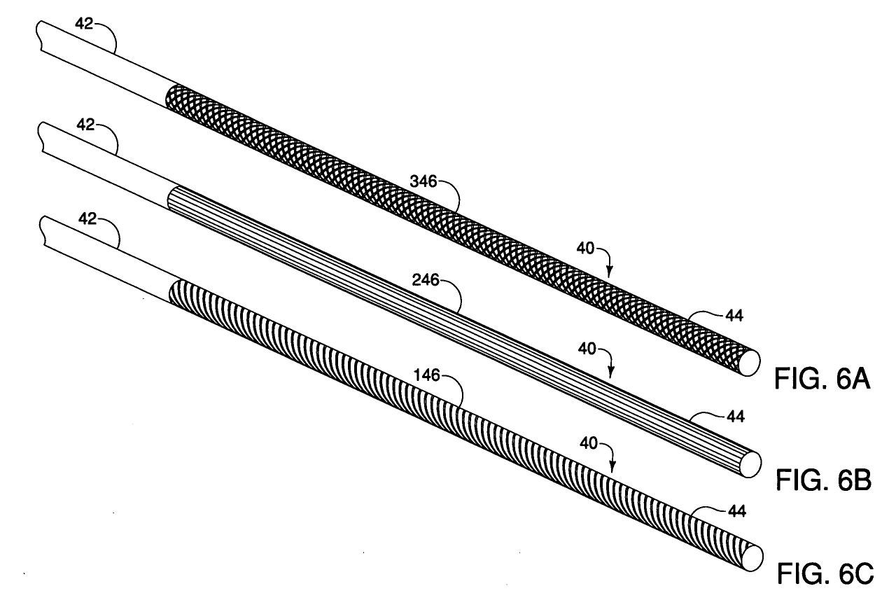

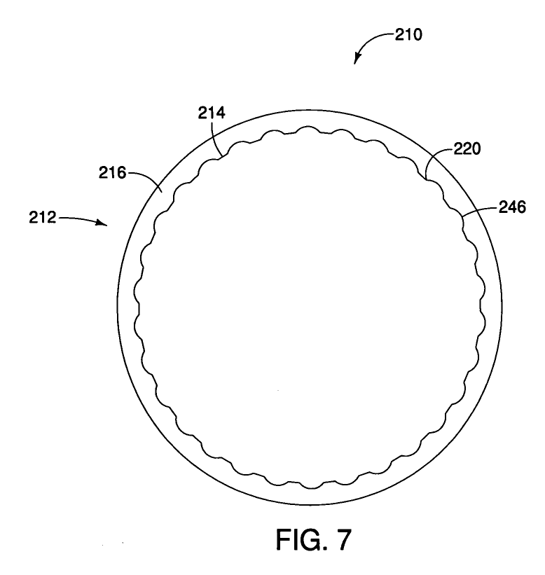

FIG. 6 shows a stent 30 is in contact with the non-uniform surface 20 of the inner tube 14. In this particular embodiment, the stent 30 is a self-expandable stent. When the stent 30 is in a compressed state, the outside surface of the stent is generally smooth, while there are points of the struts that extend outwardly. Therefore, the compressed stent 30 has a greater amount of surface area and surface contact with the inner surface 16 of the inner tube 14. The non-uniform surface 20 only has minimal interaction with the struts of the stent 30. The reduced points of contact between the stent 30 and the inner surface 16 of the inner tube 14 of the introducer sheath 12 enable an ease of movement of the stent 30 during deployment. Thus, the lessened amount of surface contact reduces the amount of friction between the inner surface 16 of the inner tube 14 and a stent during deployment. FIG. 7 demonstrates how the spiral configuration of the non-uniform surface 120 decreases the points of contact between the stent 130 and the inner surface 116 of the inner tube 114. FIG. 9 demonstrates how the non-uniform surface 220 with splines 246 decrease the points of contact between the stent 220 and the inner surface 216 of the inner tube 214. These improvements allow for easier deployment of the stent 30 to the vessel of the patient.

The present invention has been described in the context of a delivery system for the deployment of a stent within a damaged vessel. However, the present invention may provide a benefit to other medical devices. In particular, the present invention may be a benefit any time a medical device is moveably disposed through the lumen of the introducer sheath by reducing the number of contact points between the medical device and the introducer sheath.

It is therefore intended that the foregoing detailed description be regarded as illustrative rather than limiting, and that it be understood that it is the following claims, including all equivalents, that are intended to define the spirit and scope of this invention.

1. A medical device delivery system comprising:
an introducer sheath comprising:
an elongated tubular member having a passageway extending generally longitudinally therethrough, the passageway being defined by an inner surface of the tubular member and configured to receive a medical device moveably disposed therein;
an inner catheter slidably disposed within the passageway of the introducer sheath; and
a medical device disposed within a distal portion of the introducer sheath and axially supported by the inner catheter,
wherein at least a portion of the inner surface of the introducer sheath comprises a non-uniform surface configured to reduce surface contact with an outer surface of the medical device.

2. The delivery system of claim 1, wherein the inner catheter includes a plurality of positioning members disposed axially along a longitudinal axis of said inner catheter.

3. The delivery system of claim 1, wherein the tubular member of the introducer sheath comprises a wall disposed about the passageway, the wall comprising a reinforcement material extending longitudinally along at least a portion of the tubular member.

4. The delivery system of claim 3, wherein the tubular member further comprises an inner tube and an outer tube positioned longitudinally around the inner tube, where the reinforcement material is disposed between the inner tube and the outer tube.

5. The delivery system of claim 1, wherein the inner surface of the elongated tubular member includes a shrink tube.

6. The delivery system of claim 1, wherein the non-uniform surface is created by a structural element removeably engaged with the inner surface of the shrink tube.

7. The delivery system of claim 6, wherein the structural element comprises a wire braid disposed about a mandrel.

8. The delivery system of claim 6, wherein structural element comprises a wire spirally wound about a mandrel.

9. The delivery system of claim 6, wherein the structural element comprises a mandrel having an outer surface with a non-uniform pattern.

10. The delivery system of claim 1, wherein the medical device is a self-expanding stent.

11. The delivery system of claim 1, wherein the medical device is a balloon-expandable stent.

12. An introducer sheath, comprising:
an elongated tubular member having a passageway extending generally longitudinally therethrough, the passageway being defined by an inner surface of the tubular member and configured to receive a medical device moveably disposed therein;
wherein at least a portion of the inner surface comprises a non-uniform surface configured to reduce surface contact between the inner surface and the medical device.

13. The introducer sheath of claim 11, wherein the tubular member comprises a wall disposed about the passageway, the wall comprising a reinforcement material extending longitudinally along at least a portion of the tubular member.

14. The introducer sheath of claim 12, wherein the tubular member further comprises an inner tube; and an outer tube positioned longitudinally around the inner tube, where the reinforcement material is disposed between the inner tube and the outer tube.

15. The introducer sheath of claim 11, wherein the inner surface of the elongated tubular member includes a shrink tube.

16. The introducer sheath of claim 14, wherein the non-uniform surface is disposed upon an inner surface of the shrink tube.

17. The introducer sheath of claim 11, wherein the non-uniform surface is created by a structural element removeably engaged with the inner surface of the shrink tube.

18. The introducer sheath of claim 16, wherein the structural element comprises a wire braid disposed about a mandrel.

19. The introducer sheath of claim 16, wherein structural element comprises a wire spirally wound about a mandrel.

20. The introducer sheath of claim 16, wherein the structural element comprises a mandrel having an outer surface with a non-uniform pattern.

21. A method of providing non-uniform surface to an introducer sheath, comprising the steps of:
providing a mandrel having a non-uniform outer surface along at least a distal portion thereof;
disposing a tubular member over the mandrel; and
applying heat to at least one of the tubular member and the mandrel to a temperature sufficient to allow an inner surface of the tubular member to at least partially adopt the non-uniform outer surface of the mandrel

22. The method of claim 20, further comprising the step of providing a mandrel having a uniform outer surface and winding a coil wire around the mandrel to form the non-uniform surface portion.

23. The method of claim 21, wherein the coil wire has a braided configuration.

24. The method of claim 21, wherein the coil wire has a spiral configuration.

25. The method of claim 20, wherein the non-uniform outer surface of the mandrel is a knurl pattern.

26. The method of claim 20, wherein the non-uniform outer surface of the mandrel is a plurality of splines.
